(19) United States
(12) Patent Application Publication
(10) Pub. No.: US 2025/0302795 A1
(43) Pub. Date: Oct. 2, 2025

(54) SMALL MOLECULE COMPOUNDS WITH NEGATIVE REGULATORY EFFECTS ON Nrf2 SIGNALING PATHWAYS AND THEIR POTENTIAL APPLICATIONS

(71) Applicant: Beijing Hebabiz Biotechnology Co., Inc., Beijing (CN)

(72) Inventors: James Zhou, Westport, CT (US); Bai-Bo Xie, Beijing (CN)

(73) Assignee: Beijing Hebabiz Biotechnology Co., Inc., Beijing (CN)

(21) Appl. No.: 18/865,630

(22) PCT Filed: Jan. 4, 2023
(86) PCT No.: PCT/CN2023/070440

§ 371 Date: Nov. 13, 2024

(30) Foreign Application Priority Data
May 17, 2022 (CN) 202210532017.6

(51) Int. Cl. A61K 31/352; A61K 31/12; A61P 1/16

(52) U.S. Cl. CPC A61K 31/352; A61K 31/12; A61P 1/16

(57) ABSTRACT

The invention pertains to the field of medical technology and health food, in particular, specifically providing a small molecule composition that exerts negative regulation on the Nrf2 signaling pathway. This composition comprises licorice chalcone A, licorice chalcone C, licorice chalcone D, licorice chalcone E, formononetin, glabrone and Licoflavone C, which can be utilized for the prevention or treatment of non-alcoholic fatty liver disease.

### TECHNICAL FIELD

The present invention belongs to the field of medical technology and health food, specifically providing a small molecule composition that exerts negative regulation on the Nrf2 signaling pathway, which can be utilized for the negative modulation of the Nrf2 signaling pathway or for the prevention and treatment of non-alcoholic fatty liver disease.

### BACKGROUND OF THE INVENTION

Nonalcoholic fatty liver disease (NAFLD), nonalcoholic steatohepatitis (NASH), or metabolically associated steatohepatitis (MASH) is a prevalent chronic liver disease characterized by a complex pathogenesis that typically involves the following stages: dysregulation of glycolipid metabolism leading to hepatic lipid accumulation, hepatocyte fat oxidation resulting in liver inflammation, inflammation-associated fibrosis culminating in cirrhosis, and ultimately hepatocellular carcinomatosis—the development of liver cancer. Numerous studies have demonstrated that this process encompasses multiple cellular signaling pathways, with significant impact on the occurrence and progression of the disease being attributed to the activation and inhibition of the KEAP1-NRF2 signaling pathway. The NRF2 gene can activate numerous downstream genes and gene clusters, primarily functioning as an antioxidant agent while also providing protection against cellular damage and regulation of inflammatory responses. Consequently, NRF2 agonists are generally regarded as beneficial for therapeutic intervention in NAFLD or NASH.

Licorice is extensively utilized in Chinese herbal medicine and is also the most frequently employed in Chinese herbal legacy prescriptions, thus earning the title of “the king” of Chinese herbal medicine. However, within traditional Chinese medicine, licorice is typically administered as a decoction, primarily consisting of water-soluble molecules from a pharmaceutical material perspective.

Licochalcone A, the primary non-water-soluble chalcone molecule found in Licorice, acts as an agonist of the KEAP1-NRF2 pathway and is involved in regulating the Nrf2 signaling pathway, similar to other Licochalcones and chalcone molecules. However, this study discovered a glycyrrhiza extract abundant in Licochalcone A that unexpectedly exhibited negative regulatory effects on Nrf2 gene expression during the progression of non-alcoholic fatty liver disease (NAFLD). Despite its potential as an Nrf2 antagonist, further research was conducted based on extensive experience. Consequently, drugs or health products with significant therapeutic benefits for NAFLD treatment were obtained.

### SUMMARY OF THE INVENTION

The technical problem addressed by this invention is to provide innovative small molecule compositions that exert a negative regulatory effect on the Nrf2 signaling pathway, while effectively preventing or treating non-alcoholic fatty liver disease. Additionally, the invention encompasses a pharmaceutical preparation or health food product containing said small molecule composition and its application.

Specifically, in the first aspect, the present invention provides a small molecular composition for the negative regulation of the Nrf2 signaling pathway, which comprises licochalcone A, licochalcone C, licochalcone D, licochalcone E, formononetin, glabrone and Licoflavone C. This composition effectively suppresses Nrf2 gene expression during the occurrence and development of nonalcoholic fatty liver.

Among the inventions, licochalcone A, licochalcone C, licochalcone D, licochalcone E, formononetin, glabrone and licoflavine C can be derived from either botanical sources or synthesized chemically. Ideally, the preferred source is Glycyrrhiza inflata Batalin.

Preferably, in the pharmaceutical composition of the first aspect of the present invention, the weight ratio of licochalcone A, licochalcone C, licochalcone D, licochalcone E, formononetin, glabrone and licoflavine C is 60˜75:2˜6:1˜4:5˜10:1˜5:1˜5:1˜5.

More preferably, in the pharmaceutical composition of the first aspect of the present invention, the weight ratio of licochalcone A, licochalcone C, licochalcone D, licochalcone E, formononetin, glabrone and licoflavine C is 65˜75:3˜5:1˜3:6˜8:2˜3:2˜3:1˜2.

In the second aspect, the present invention provides preparations, comprising compositions of the first aspect along with pharmaceutically or food-acceptable excipients. These preparations can be categorized as either pharmaceutical or health food products.

In this context, the term “pharmaceutically acceptable excipients” encompasses carriers, diluents, and other excipients that are compatible with the active ingredient of the drug. The utilization of pharmaceutically acceptable excipients in pharmaceutical preparations is well-established among professionals in the field. The pharmaceutical preparation of this invention comprises niacinamide mononucleotide, hifrucin, and erythritol as active ingredients. These active ingredients are combined with pharmaceutically acceptable adjuvants (such as carriers, excipients, diluents, etc., well known to ordinary technicians in the field) to prepare various formulations. Preferably, solid and liquid preparations such as tablets, pills, capsules (including sustained release or delayed release forms), powders, suspensions, granules, syrups, emulsions, suspensions and various slow-release dosage forms, preferably for oral administration. In one specific embodiment of the invention, the composition of the first aspect is diluted into a liquid preparation using a 0.5% CMC-Na solution. According to the composition of the first aspect, the effective dosage for prevention or treatment can be determined based on experimental animal models.

In this context, the term “acceptable excipients on food” encompasses suitable carriers, excipients, diluents, flavoring agents, colorants, and other additives that are compatible with the active ingredients for promoting food health. Compositions containing niacinamide mononucleotides, momorin and erythroitol can be directly incorporated or added to food or its raw materials through methods such as coating or blending with other food products.

In the third aspect, the present invention provides the application of the composition of the first aspect for preparing reagents that negatively regulate the Nrf2 signaling pathway. Accordingly, In the fourth aspect, the present invention provides a method for negative regulation of the Nrf2 signaling pathway by utilizing the said composition of the first aspect.

In the fifth aspect, the present invention provides the application of the composition of the first aspect for the preparation of drugs intended for preventing or treating non-alcoholic fatty liver disease (NAFLD). Consequently, in the sixth aspect, the present invention provides a method for preventing or treating non-alcoholic fatty liver disease, which involves administering the composition described in the first aspect.

In this context, the application object can be either a human or an experimental animal, with a preference for humans.

Preferably, the non-alcoholic fatty liver disease is a condition characterized non-alcoholic steatohepatitis (NASH).

More preferably, the non-alcoholic fatty liver disease is characterized by dysregulation in glycolipid metabolism and can lead to liver fibrosis, cirrhosis, or hepatocellular carcinoma.

For the purpose of facilitating comprehension, the present invention is elaborated upon in specific embodiments and accompanied by illustrations. It should be emphasized that these descriptions serve as mere illustrative depictions and do not impose any limitations on the scope of the present invention. Based on the content provided in this specification, numerous modifications and alterations to the invention would be readily apparent to those skilled in the relevant field.

### BRIEF DESCRIPTION OF THE DRAWINGS

FIG. 1 shows the characteristic and control maps of NR218 extracts.

FIG. 2 shows the chemical structure of the primary constituents found in NR218 extract.

FIG. 3 shows the results of HE staining in rat liver tissues (200× 400×), in which (A) Normal control group; (B) Model group; (C) Bicyclol positive control group; (D) Lovastatin positive control group; (E) Ocaliva positive control group; (F) NR218 low-dose group; (G) NR218 medium-dose group; (H) NR218 high-dose group.

FIG. 4 shows the staining results of oil red in rat liver tissues (200× 400×), among which (A) Normal control group; (B) Model group; (C) Bicyclol positive control group; (D) Lovastatin positive control group; (E) Ocaliva positive control group; (F) NR218 low-dose group; (G) NR218 medium-dose group; (H) NR218 high-dose group.

FIG. 5 shows the effects of NR218 on the expression of AMPK, NF-κB, Nrf2 and SIRT1 proteins in rat liver tissues with nonalcoholic steatohepatitis (400×), among which (A) Normal control group; (B) Model group; (C) NR218 low-dose group; (D) NR218 medium-dose group; (E) NR218 high-dose group.

FIG. 6 shows the results of HE staining in rat liver tissues (200× 400×), among which (A) Normal control group; (B) Model group; (C) Ocaliva positive control group; (D) Lovastatin positive control group; (E) Bicyclol positive control group; (F) NR218 high-dose group; (G) NR218 medium-dose group; (H) NR218 low-dose group.

FIG. 7 shows the staining results of oil red in rat liver tissues (200× 400×), among which (A) Normal control group; (B) Model group; (C) Ocaliva positive control group; (D) Lovastatin positive control group; (E) Bicyclol positive control group; (F) NR218 high-dose group; (G) NR218 medium-dose group; (H) NR218 low-dose group.

FIG. 8 shows the Masson staining results in rat liver tissues (200× and 400×), among which (A) Normal control group; (B) Model group; (C) Ocaliva positive control group; (D) Lovastatin positive control group; (E) Bicyclol positive control group; (F) NR218 high-dose group; (G) NR218 medium-dose group; (H) NR218 low-dose group.

FIG. 9 shows the effect of NR218 on the expression of NF-κB, Nrf2, Smad, TGF-β, α-SMA, AMPK, NS5ATP9 and SIRT1 proteins in rat liver tissues with nonalcoholic steatohepatitis, among which (A) Normal control group; (B) Model group; (C) NR218 low-dose group; (D) NR218 medium-dose group; (E) NR218 high-dose group.

### DETAILED DESCRIPTION

The following embodiments illustrate the content of the present invention. Unless otherwise specified, the technical means used in the embodiments are conventional means well-known to those skilled in the art and commercially available common instruments and reagents, and the manufacturer's instructions for the corresponding instruments and reagents can be referred to.

### Embodiment 1 Preparation of Licorice Extract

The roots and rhizomes of Glycyrrhiza were extracted, crushed, soaked in water at 60° C., and the resulting extract was treated separately. The residue was dried to a water content of (15±5) %, then extracted by reflux with ethanol at a concentration of (85±5) %. The ethanol extract was combined with the extraction solution, recovered and concentrated to the appropriate amount. Subsequently, it was added to a macroporous adsorption resin column that had been treated beforehand. Elution was performed using different concentrations of ethanol sequentially, collecting the corresponding eluent each time. The ethanol was then recovered and concentrated to obtain the desired amount before being added to a polyamide resin column that had also been treated beforehand. Elution with different concentrations of ethanol followed again, collecting the corresponding eluent each time. Finally, the ethanol was recovered and concentrated into a thick paste which underwent pressure drying and crushing process for obtaining NR218 component (extract). This particular component exhibited interesting properties during preliminary testing, therefore an in-depth study on it has been conducted.

### Embodiment 2 Identification of the NR218 Extract

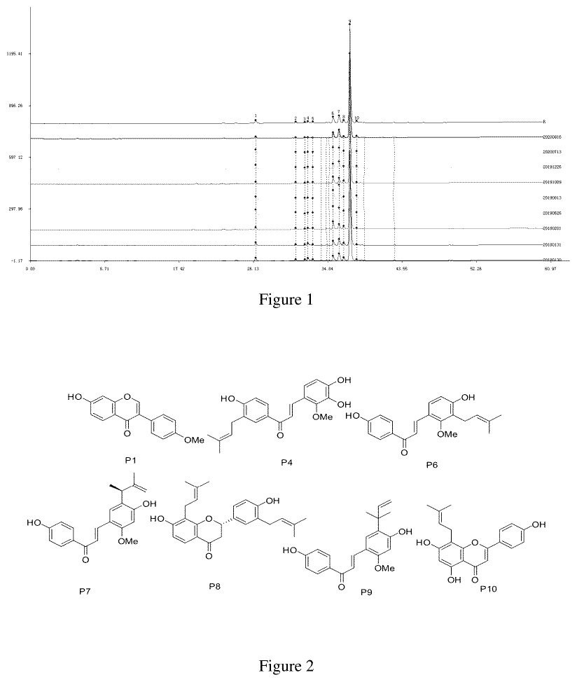

After conducting commissioned identification, the characteristic spectrum of NR218 extract is presented in FIG. 1. The structure analysis of the seven most significant compounds is depicted in FIG. 2, namely licochalcone A, licochalcone C, licochalcone D, licochalcone E, formononetin, glabrone and licoflavone C. Their respective proportions are displayed in Table 1. Additionally, HPLC-MS detection revealed the presence of minor components as indicated in Table 2.

TABLE 1
Primary constituents and their respective ratios in NR218 extract

| Peak Number | P1 | P4 | P6 | P7 | P8 | P9 | P10 |
|---|---|---|---|---|---|---|---|
| Chemical Name | Formononetin | Licochalcone | Licochalcone | Licochalcone | Glabrone | Licochalcone | Licoflavone |
| | | D | C | E | | A | C |
| Peak Area (%) | 2.52 | 2.21 | 3.80 | 6.75 | 2.76 | 72.04 | 1.24 |

TABLE 2
Other constituents in NR218 extract

| | Molecular | | |
|---|---|---|---|
| No. | Weight | MS (m/z) | Chemical Name |
| 1 | 286 | 287[M + H] | Licochalcone B |
| 2 | 270 | 271[M + H] | Echinatin |
| 3 | 256 | 257[M + H] | Isoliquiritigenin |
| 4 | 256 | 257[M + H] | Liquiritigenin |
| 5 | 422 | 423[M + H] | Licoisoflavanone B |
| 6 | 422 | 423[M + H] | Licoisoflavanone C |
| 7 | 322 | 323[M + H], | Licoflavone A |
| | | 645[2M + H], | |
| | | 667[2M + Na] | |
| 8 | 354 | 355[M + H], | Licoisoflavone A |
| | | 731[2M + Na] | |
| 9 | 352 | 353[M + H] | Licoisoflavone B |
| 10 | 320 | 321[M + H] | Alpinumisoflavone |
| 11 | 336 | 337[M + H] | Glabrone [M + H] |
| 12 | 390 | 391[M + H] | Licoflavone B |
| 13 | 388 | 389[M + H] | Licoflavone E |
| 14 | 388 | 389[M + H] | Kanzonol E |
| 15 | 470 | 471[M + H] | Glycyrrhetic acid |
| 16 | 468 | 469[M + H] | 3-Oxo-glycyrrhetic acid |
| 17 | 358 | 358[M] | Gancaonin O |
| 18 | 512 | 512[M] | 3-β-Acetylglycyrrhetic acid |

### Embodiment 3 Preparation of the NR218 Composition

In order to ensure product stability, a mixture of licochalcone A (7.2 g), licochalcone C (0.38 g), licochalcone D (0.22 g), licochalcone E (0.68 g), formononetin (0.25 g), glabrone (0.28 g) and licoflavone C (0.12 g) was prepared as NR218 compositions for further testing.

### Embodiment 4 Effect and Therapeutic Efficacy of NR218 on NRF2 Pathway in Rat Model of Non-Alcoholic Steatohepatitis Induced by High-Fat Diet

### 1. Materials and Methods

Mold feed: High-fat feed (88% base feed +10% lard +2% cholesterol) was purchased from SPF (Beijing) Biotechnology Co., Ltd., with license number SCXK (Beijing) 2019-0010 and certificate number 1103242000022188.

Animal: SPF-grade male Wistar rats weighting 160±10 g were purchased from SPF (Beijing) Biotechnology Co., Ltd., with license number SCX (Beijing) 2019-0010.

Drug: NR218 composition; Bicyclol tablets were obtained from Beijing Union Pharmaceutical Factory, with a specification of 50 mg per tablet and lot number 191116; Lovastatin capsules were obtained from Yangtze River Pharmaceutical Group, with a specification of 20 mg per capsule and lot number 19030461; Ocaliva was obtained from Hubei Jiuzhou Kangda Biotechnology Co., Ltd., with a specification of 100 g per bag and lot number 20200210.

Instrument: Multifunctional enzyme marker, Model H1M, was provided by Guangzhou Darui Biotechnology Co., Ltd.; Multi-tissue homogenizer, Model Tissuelyser-24, was provided by Shanghai Jingxin Industrial Development Co., Ltd.; Refrigerated centrifuge, Model Micro 21R; Centrifuge, Model LR58495, was provided by Thermo Fisher Scientific; Electronic balance, Model YP10001, was provided by Shanghai Yueping Electronic Balance; Analytical Balance, Model PL602-L, was provided by Mettler Toledo; Vertical Pressure Steam Sterilizer, Model SN510C, was provided by Chongqing Amato Technology Co., Ltd.; Electrophoresis instrument, Model DYY-6C, was provided by Beijing Liuyi Instrument Factory; Protein electrophoresis and Transfer System, Model Mini-PROTEAN, was provided by BIO-RAD Corporation.

Materials: High-density lipoprotein cholesterol (HDL-C) test kit, Item No. A112-1-1 and Lot No. 20201012, was provided by Nanjing Jiancheng Bioengineering Institute; Low-density lipoprotein cholesterol (LDL-C) test kit, Item No. A113-1-1 and Lot No. 20201012, was provided by Nanjing Jiancheng Bioengineering Institute; Free fatty acid (NEFA) test kit, Item No. A042-2-1 and Lot No. 20201012, was provided by Nanjing Jiancheng Bioengineering Institute; Glutamic-oxalic Transaminase (AST/GOT) test kit, Item No. C010-2-1 and Lot No. 20201012, was provided by Nanjing Jiancheng Bioengineering Institute; Glutamic-pyruvic Transaminase (ALT/GPT) test kit, Item No. C009-2-1 and lot No. 20201012, was provided by Nanjing Jiancheng Bioengineering Institute; Total Cholesterol (TC) test kit, Item No. A111-1-1 and Lot No. 20200815, was provided by Nanjing Jiancheng Bioengineering Institute; Triglyceride (TG) test kit, Item No. A110-1-1 and Lot No. 20200814, was provided by Nanjing Jiancheng Bioengineering Institute; Uric acid (UA) test kit, Item No. C012-2-1 and Lot No. 20201012, was provided by Nanjing Jiancheng Bioengineering Institute; Blood Glucose (FPG) test kit, Item No. SH152W and Lot No. 20201012, was provided by G-clone Biotechnology Co., Ltd.; Rat Fasting Insulin (FINS) ELISA kit, Item No. SEKR-0160 and Lot No. 20201012, was provided by G-clone Biotechnology Co., Ltd.; High-efficiency RIPA Tissue Rapid Lysate, Item No. R0010 and Lot No. 20200926, was provided by Solarbio Science & Technology Co., Ltd.; BCA Kit, Item No. PC0020 and Lot No. 20201010, was provided by Solarbio Science & Technology Co., Ltd.; SIRT1 Antibody, Item No. 9475S, was provided by Cell Signaling technology Co., Ltd.; AMPKα1 Antibody, Item No. 10929-2-AP, was provided by Proteinteck; NF-κB p65 Antibody, Item No. 10745-1-AP, was provided by Proteinteck Inc.; Nrf2 Antibody, Item No. 16396-1-AP, was provided by Proteinteck Inc.; GAPDH Antibody, Item No. 10494-1-AP, was provided by Proteinteck Inc.

### 2. Methods

### (1) Establishment of a Non-Alcoholic Steatohepatitis Model in Rats Induced by High-Fat Diet and Administration of Drugs

A total of one hundred and twenty male Wistar rats (SPF grade, weighing 160±10 g) were randomly assigned to eight groups, including a normal control group, model group, Bicyclol group, Lovastatin group, Ocaliva group, and NR218 high (90 mg/kg), medium (30 mg/kg), and low (10 mg/kg) dose groups with fifteen rats in each. The normal control group was fed a standard diet for twelve weeks while the other groups were fed a high-fat diet consisting of 88% basal diet+10% lard+2% cholesterol for twelve weeks. After establishing the model, the drug groups received their respective treatments at a dosage of 10 ml/kg while the normal and model groups received an equal volume of 0.5% CMC-Na solution. The rats were provided with ad libitum access to water and food, while the animal chamber was maintained under controlled conditions of quietness, natural lighting, a temperature of 25±0.5° C., and a humidity level of 55±5%. The rats' body weights were measured once per week and recorded.

### (2) Collection and Analysis of Samples

### \textcircled{1} Survey on the General Condition

The rats were weighed weekly to monitor the fluctuations in body weight within each experimental group.

\textcircled{2} Revealing Serum Biochemical Markers Associated with Nonalcoholic Steatohepatitis

At the end of the 8th week of administration, all rats were anesthetized with a 20% ulinastatin solution (10 ml/kg) via an intraperitoneal injection after the final administration. Subsequently, blood was collected from the abdominal aorta post-anesthesia. After standing for 1 hour, the blood was centrifuged at 4° C. and 3000 r/min for 15 minutes to obtain the upper serum fraction which was then stored at −80° C. Following the instructions provided by the biochemical index detection kit, levels of HDL-C, LDL-C, NEFA, AST, ALT, UA, TC, TG, FPG and FINS in rat serum were determined. Additionally, insulin resistance index [(HOMA-IR)=FPG (mmol/L)×FINS (mIU/L)/22.5] was assessed.

\textcircled{3} Liver indices was determined by dividing the wet weight of the liver by the body weight of rats and multiplying by 100%, after liver tissue was isolated, washed with normal saline, dried using filter paper, and weighed following blood sampling from the abdominal aorta.
\textcircled{4} HE staining and oil red staining were performed on liver tissue samples obtained from the abdominal aorta of rats. The tissue was fixed in 10% neutral formalin, followed by embedding, slicing, dewaxing, staining, transparency treatment, and sealing using routine procedures. Microscopic examination was conducted to observe the pathological changes in rat liver tissues across different groups. Additionally, the NASH-CRN scoring system was employed to assess NAS scores.
\textcircled{5} Detection of Proteins Associated with Non-Alcoholic Steatohepatitis

Immunohistochemical staining: Liver tissue sections, embedded in paraffin and cut to a thickness of 5 μm, were incubated with the target antibody for 90 minutes. Subsequently, they were treated with peroxidase-conjugated secondary antibody for 30 minutes. Following this, the sections were exposed to the streptavidin-peroxidase-biotin complex at room temperature for 20 minutes. After chromogenic staining, the liver tissue sections were observed and analyzed using a microscope.

For the Western blotting experiment, appropriate liver tissue was obtained and digested using a high-efficiency RIPA tissue rapid cleavage solution and PMSF for efficient protein extraction. The extracted liver tissue protein was quantitatively assessed using the BCA Protein Assay Kit. Following the standard procedure of Western blotting, changes in protein expression levels of AMPK, SIRT1, NF-κB, and Nrf2 were detected with GAPDH serving as an internal reference. Quantitative analysis was performed using ImageJ 1.48 software.

### (3) Statistical Analysis and Processing

The results were expressed as mean±standard deviation (X̅±SD). One-way analysis of variance was performed using SPSS software version 17.0. Statistical significance was considered at P<0.05 and P<0.01 levels. GraphPad Prism software version 6.02 was utilized for data visualization.

### 3. Results

(1) Alterations in the Body Weight of Rat Cohorts within Each Experimental Group

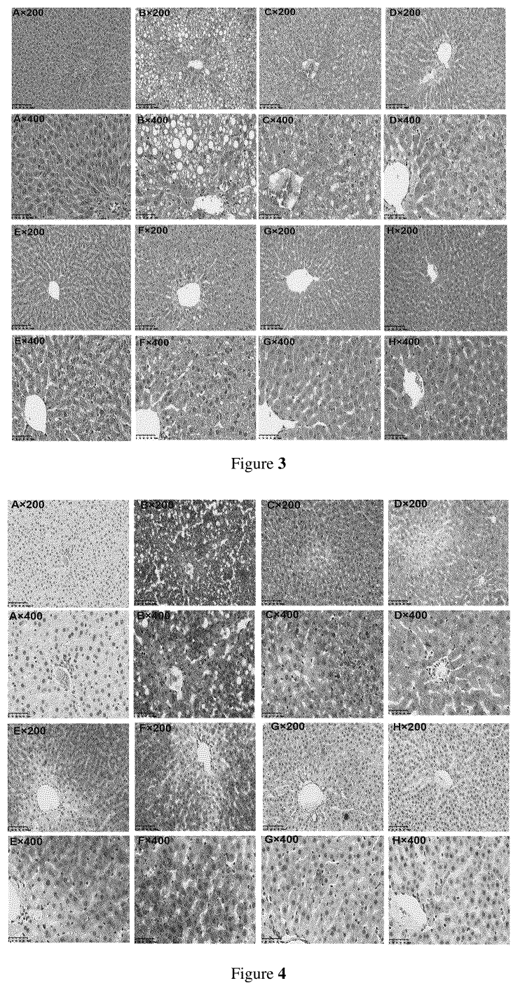

As shown in FIG. 3, the results of the weight analysis revealed an increasing trend in rat weights during the 12th week prior to modelling. Following administration, there was a slight decrease in weight observed across all groups. However, no significant differences were observed.

TABLE 3
Changes in body weight of rats in each group (X̅ ± SD, n = 15)

| | | | | | | NR218 | NR218 | NR218 |
|---|---|---|---|---|---|---|---|---|
| | Normal | | | | | low- | medium- | high- |
| Time | control | Model | Bicyclol | Lovastatin | Ocaliva | dose | dose | dose |
| (weekly) | group | group | group | group | group | group | group | group |
| 0 | 196.58 ± 4.82 | 198.02 ± 2.88 | 195.09 ± 3.95 | 196.21 ± 4.57 | 197.61 ± 4.50 | 195.68 ± 5.72 | 197.21 ± 3.58 | 196.82 ± 5.44 |
| 1 | 259.19 ± 10.82 | 263.14 ± 6.40 | 254.99 ± 12.09 | 258.64 ± 9.56 | 257.27 ± 10.59 | 258.83 ± 15.85 | 258.49 ± 8.59 | 259.35 ± 12.87 |
| 2 | 312.61 ± 22.25 | 327.50 ± 13.74 | 307.37 ± 12.92 | 315.91 ± 19.58 | 318.89 ± 11.61 | 324.79 ± 24.11 | 322.35 ± 16.33 | 315.70 ± 22.27 |
| 3 | 364.23 ± 17.34 | 366.71 ± 21.41 | 343.86 ± 17.21 | 356.16 ± 26.94 | 362.58 ± 18.50 | 362.01 ± 26.66 | 362.48 ± 23.97 | 353.50 ± 29.98 |
| 4 | 397.15 ± 19.10 | 404.45 ± 25.49 | 379.51 ± 22.29 | 392.60 ± 32.93 | 403.03 ± 23.75 | 396.86 ± 30.04 | 400.09 ± 31.07 | 388.87 ± 37.38 |
| 5 | 433.01 ± 18.45 | 445.06 ± 32.71 | 413.81 ± 26.94 | 432.81 ± 38.14 | 439.43 ± 29.45 | 437.74 ± 35.38 | 437.61 ± 36.59 | 433.16 ± 45.05 |
| 6 | 465.29 ± 24.47 | 471.21 ± 42.63 | 444.77 ± 32.35 | 464.35 ± 45.01 | 474.15 ± 35.23 | 470.72 ± 39.50 | 471.42 ± 41.56 | 456.47 ± 46.51 |
| 7 | 486.75 ± 30.96 | 492.39 ± 42.83 | 461.64 ± 35.88 | 482.88 ± 44.36 | 492.08 ± 37.59 | 487.11 ± 39.22 | 489.89 ± 46.76 | 474.01 ± 50.09 |
| 8 | 514.11 ± 32.49 | 517.88 ± 48.22 | 493.67 ± 41.04 | 503.37 ± 45.95 | 520.77 ± 41.63 | 517.71 ± 41.03 | 516.21 ± 53.09 | 490.44 ± 57.42 |
| 9 | 536.47 ± 37.02 | 543.48 ± 45.93 | 510.27 ± 41.17 | 530.26 ± 48.68 | 548.10 ± 47.45 | 540.46 ± 47.52 | 539.85 ± 56.88 | 523.13 ± 56.01 |
| 10 | 541.64 ± 33.91 | 553.75 ± 44.24 | 526.93 ± 47.38 | 548.47 ± 45.65 | 552.73 ± 51.34 | 560.88 ± 51.88 | 554.09 ± 64.12 | 529.78 ± 63.56 |
| 11 | 554.64 ± 35.47 | 570.19 ± 50.78 | 541.47 ± 50.20 | 547.23 ± 54.49 | 552.52 ± 56.26 | 576.59 ± 58.96 | 564.06 ± 66.57 | 546.73 ± 61.07 |
| 12 | 560.67 ± 30.87 | 581.74 ± 52.28 | 550.05 ± 58.70 | 543.06 ± 66.86 | 570.97 ± 53.36 | 584.69 ± 52.67 | 593.09 ± 72.45 | 551.62 ± 59.03 |
| 13 | 549.70 ± 29.05 | 578.25 ± 57.61 | 526.35 ± 68.66 | 534.63 ± 62.63 | 552.25 ± 68.34 | 580.35 ± 60.62 | 581.47 ± 72.21 | 532.73 ± 51.39 |
| 14 | 543.69 ± 33.67 | 560.62 ± 66.34 | 515.27 ± 58.62 | 542.19 ± 52.03 | 551.61 ± 63.47 | 541.49 ± 71.98 | 554.29 ± 56.00 | 517.17 ± 50.14 |
| 15 | 539.88 ± 37.07 | 556.49 ± 63.56 | 519.07 ± 54.38 | 543.36 ± 47.44 | 559.94 ± 61.90 | 531.76 ± 67.92 | 554.37 ± 47.90 | 524.96 ± 58.86 |
| 16 | 556.63 ± 44.18 | 557.60 ± 76.06 | 539.33 ± 58.40 | 547.25 ± 37.26 | 572.35 ± 60.86 | 550.64 ± 72.41 | 576.88 ± 45.61 | 540.63 ± 59.02 |
| 17 | 572.43 ± 42.99 | 577.99 ± 73.13 | 562.79 ± 61.59 | 561.64 ± 53.62 | 573.51 ± 72.23 | 559.50 ± 71.69 | 588.91 ± 52.43 | 555.21 ± 60.18 |
| 18 | 596.53 ± 41.21 | 590.15 ± 69.93 | 588.75 ± 70.94 | 593.39 ± 48.55 | 599.13 ± 55.71 | 556.26 ± 79.60 | 618.68 ± 58.15 | 569.45 ± 74.85 |
| 19 | 622.28 ± 43.38 | 610.74 ± 69.58 | 593.71 ± 72.81 | 615.74 ± 52.23 | 615.97 ± 56.23 | 588.95 ± 72.52 | 649.76 ± 65.74 | 582.75 ± 87.92 |
| 20 | 609.72 ± 38.28 | 591.66 ± 73.28 | 586.53 ± 112.38 | 600.22 ± 50.84 | 601.19 ± 60.01 | 606.71 ± 68.67 | 640.14 ± 65.91 | 563.59 ± 95.66 |

### (2) Effects of NR218 on a Hepatic Index in a Rat Model of Nonalcoholic Steatohepatitis

As shown in Table 4, after the experiment, through the investigation of the liver indices in each group, it was observed that the model group exhibited a significant increase compared to the normal control group (P<0.01). Conversely, treatment with Bicyclol, Lovastatin, Ocaliva (positive controls), and NR218 at high, medium, and low doses resulted in a significant decrease compared to the model group (P<0.01 or P<0.05). Notably, NR218 at medium and high doses demonstrated superior efficacy compared to positive drugs.

TABLE 4
Liver indices of rats in each group (X̅ ± SD)

| Groups | Sample size | Liver Indices (%) |
|---|---|---|
| Normal control group | 15 | 2.48 ± 0.48 |
| Model group | 15 | 4.37 ± 1.12^{##} |
| Bicyclol group | 15 | 3.77 ± 1.17* |
| Lovastatin group | 15 | 3.23 ± 0.56** |
| Ocaliva group | 15 | 2.95 ± 0.68** |
| NR218 low-dose group | 15 | 3.21 ± 0.75** |
| NR218 medium-dose group | 15 | 2.27 ± 0.32** |
| NR218 high-dose group | 15 | 2.57 ± 0.83** |

Note:
^{##}P < 0.01 compared to the normal control group; **P < 0.01 and *P < 0.05 compared to the model group.

(3) Effects of NR218 on the Hepatic Histopathology in Rats with Nonalcoholic Steatohepatitis

As shown in FIG. 3, the HE staining results revealed liver cells in the normal control group exhibited well-organized with a clear structure of liver lobules, no steatosis, and absence of inflammatory cell infiltration. In contrast, the model group displayed swollen liver cells containing lipid droplets of varying sizes, marginalized nuclei, balloon-like degeneration, and inflammatory cell infiltration. Furthermore, all treatment groups showed alleviated steatosis to some extent along with reduced inflammatory cells. Notably, the NR218 high-dose group exhibited the most pronounced effects with nearly normal morphology of liver cells.

As shown in FIG. 4, the Oil red O staining results revealed a significant increase in hepatic fat content in the model group, accompanied by an irregular arrangement of hepatocytes and loss of cytoskeletal integrity. In this group, hepatocytes exhibited predominantly polycystic bulla lipid droplets that were distributed mainly in sheets, often congregating on the surface of the tissue slice after sealing. Conversely, rats treated with NR218 displayed scattered small lipid droplets within hepatocytes, demonstrating a remarkable improvement in both steatosis severity and lipid droplet accumulation compared to the model group.

(4) Effects of NR218 on Serum Biochemical Parameters in Rats with Nonalcoholic Steatohepatitis

According to the instructions of the biochemical index detection kit, we measured the levels of serum ALT, AST, TC, TG, HDL-C, LDL-C, UA, NEFA, FPG and FINS in rats. The experimental results demonstrated in Table 5 that rats in the model group compared to the normal control group, exhibited significantly elevated levels of serum ALT, AST, TC, TG LDL-C, UA and NEFA (P<0.01 or P<0.05), along with a significant decrease in HDL-C level (P<0.01). In comparison to the model group, NR218 significantly reduced serum AST, ALT, TC, TG, LDL-C, UA and NEFA levels while simultaneously increasing HDL-C content (P<0.01 or P<0.05), thereby improving liver function and lipid metabolism in non-alcoholic steatohepatitis rats. By evaluating the insulin resistance index (HOMA-IR) among rat groups, it was observed that HOMA-IR significantly increased in rats from the model group when compared to those from the normal control group (P<0.01). However, NR218 treatment substantially alleviated insulin resistance in nonalcoholic steatohepatitis rats as compared to those from the model group. The above findings indicate that NR218 exhibits significant hepatoprotective effects by improving liver function, reducing hepatic lipid accumulation, and ameliorating insulin resistance among non-alcoholic steatohepatitis rats.

TABLE 5
Effects of NR218 on serum biochemical indices of rats with non-alcoholic steatohepatitis (X̅ ± SD, n = 15)

| | ALT | AST | TC | TG | HDL-C | LDL-C | UA | NEFA | HOMA- |
|---|---|---|---|---|---|---|---|---|---|
| Groups | (U/L) | (U/L) | (mmol/L) | (mmol/L) | (mmol/L) | (mmol/L) | (mol/L) | (mmol/L) | IR |
| Normal | 646.54 ± | 19.75 ± | 2.11 ± | 0.65 ± | 1.92 ± | 0.81 ± | 99.79 ± | 0.77 ± | 1.03 ± |
| control | 137.29 | 4.34 | 0.43 | 0.28 | 0.29 | 0.23 | 35.47 | 0.26 | 0.50 |
| group | | | | | | | | | |
| Model | 789.84 ± | 24.49 ± | 3.75 ± | 1.18 ± | 1.18 ± | 1.13 ± | 154.64 ± | 1.13 ± | 1.65 ± |
| group | 267.04^{#} | 3.97^{#} | 1.23^{##} | 0.48^{##} | 0.51^{##} | 0.37^{#} | 63.97^{##} | 0.29^{##} | 0.41^{##} |
| Bicyclol | 547.14 ± | 19.65 ± | 2.76 ± | 0.83 ± | 1.34 ± | 0.91 ± | 81.27 ± | 0.92 ± | 1.04 ± |
| group | 106.13** | 4.59** | 0.75* | 0.37** | 0.39 | 0.44 | 25.37** | 0.24* | 0.48** |
| Lovastatin | 561.25 ± | 21.43 ± | 3.09 ± | 1.02 ± | 1.43 ± | 0.96 ± | 95.77 ± | 1.05 ± | 1.23 ± |
| group | 137.85** | 4.53 | 1.02 | 0.30 | 0.61 | 0.50 | 28.99** | 0.21 | 0.58* |
| Ocaliva | 569.24 ± | 18.30 ± | 2.90 ± | 0.90 ± | 1.59 ± | 0.85 ± | 91.15 ± | 0.91 ± | 1.05 ± |
| group | 124.62** | 5.90** | 1.54* | 0.41** | 0.70* | 0.36* | 29.81** | 0.29* | 0.55** |
| NR218 | 544.14 ± | 15.82 ± | 2.42 ± | 0.82 ± | 1.82 ± | 0.73 ± | 79.10 ± | 0.93 ± | 1.31 ± |
| low- | 176.09** | 4.61** | 2.06** | 0.38** | 0.50** | 0.35** | 31.61** | 0.15* | 0.58 |
| dose | | | | | | | | | |
| group | | | | | | | | | |
| NR218 | 532.95 ± | 18.03 ± | 2.36 ± | 0.83 ± | 2.07 ± | 0.75 ± | 94.81 ± | 0.87 ± | 1.16 ± |
| medium- | 141.10** | 5.37** | 0.74** | 0.23** | 0.48** | 0.31** | 37.33** | 0.25** | 0.47** |
| dose | | | | | | | | | |
| group | | | | | | | | | |
| NR218 | 561.08 ± | 15.06 ± | 1.71 ± | 0.78 ± | 1.87 ± | 0.86 ± | 86.02 ± | 0.88 ± | 1.12 ± |
| high- | 136.58** | 6.44** | 0.29** | 0.45** | 0.56** | 0.34* | 24.09** | 0.288* | 0.508** |
| dose | | | | | | | | | |
| group | | | | | | | | | |

Note:
Compared to the normal control group, denoted by ^{#}P < 0.05 and ^{##}P < 0.01;
Compared to the model group, denoted by *P < 0.05 and **P < 0.01

(5) Effects of NR218 on the Expression of Relevant Proteins in Hepatic Tissue of Rats with Non-Alcoholic Steatohepatitis

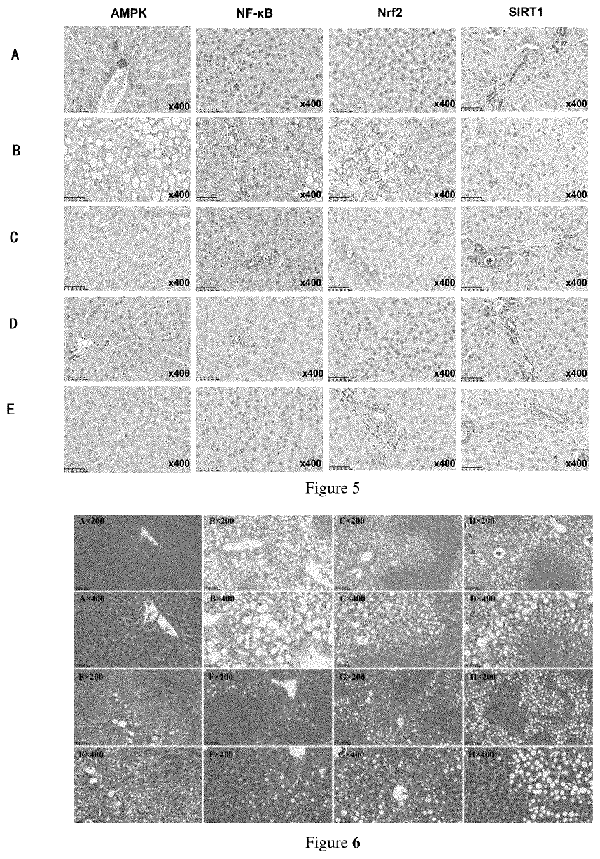

The protein expressions of AMPK, NF-κB, Nrf2, and SIRT1 in the liver tissue of rats with non-alcoholic steatohepatitis were assessed using immunohistochemical analysis. As shown in FIG. 5, the results demonstrated that treatment with high, medium, and low doses of NR218 significantly reduced the protein expression levels of NF-κB and Nrf2 to varying degrees in the liver tissue of rats with nonalcoholic steatohepatitis, while significantly increasing the protein expression levels of AMPK and SIRT1 in the liver tissue. Furthermore, as shown in Table 6, the model group exhibited a significant decrease in protein expression levels of AMPK and SIRT1 (P<0.01), while there was a significant increase in protein expression levels of NF-κB and Nrf2 (P<0.01), which compared to the normal control group. In contrast to the model group, treatment with high, medium, and low doses of NR218 significantly elevated protein expression levels of AMPK and SIRT1 (P<0.01 or P<0.05), while inhibiting protein expression levels of NF-κB and Nrf2 (P<0.01 or P<0.05), with the high dose demonstrating superior efficacy. The above findings suggest that NR218 may potentially modulate inflammation and oxidative stress in the treatment of nonalcoholic steatohepatitis through the AMPK/SIRT1/NF-κB signaling axis.

TABLE 6
Effects of NR218 on the expression levels of AMPK, SIRT1, Nrf2 and NF-
κB in hepatic tissues of rats (X̅ ± SD)

| Groups | Sample size | AMPK | SIRT1 | NRF2 | NF-κB |
|---|---|---|---|---|---|
| Normal control group | 3 | 1.00 ± 0.20 | 1.00 ± 0.12 | 1.00 ± 0.03 | 1.00 ± 0.21 |
| Model group | 3 | 0.55 ± 0.033^{#} | 0.52 ± 0.24^{##} | 2.12 ± 0.37^{##} | 2.66 ± 0.19^{##} |
| NR218 low-dose group | 3 | 0.82 ± 0.07 | 0.85 ± 0.02* | 1.24 ± 0.02** | 2.32 ± 0.21 |
| NR218 medium-dose group | 3 | 1.17 ± 0.16** | 1.06 ± 0.08** | 1.03 ± 0.20** | 1.56 ± 0.05** |
| NR218 high-dose group | 3 | 1.28 ± 0.16** | 1.04 ± 0.10** | 1.03 ± 0.13** | 1.40 ± 0.38** |

Note:
Compared to the normal control group, denoted by ^{##}P < 0.01; Compared to the model group, denoted by *P < 0.05 and **P < 0.01.

### Experimental Conclusion

The present study utilized a high-fat diet to establish a rat model of nonalcoholic steatohepatitis (NASH). It was observed that NR218 effectively ameliorated hepatic steatosis, reduced liver index, improved liver function and lipid metabolism, and decreased blood lipid levels and insulin resistance index in NASH rats. At the protein level, NR218 significantly upregulated the expression of AMPK and SIRT1 proteins while downregulating NF-κB and Nrf2 proteins in the liver. These findings suggest that NR218 exerts its beneficial effects on high-fat diet-induced NASH by modulating inflammation and antioxidant stress through the AMPK/SIRT1/NF-κB signaling axis.

### Embodiment 5: Inhibition and Therapeutic Efficacy of NR218 on NRF2 Pathway in a Rat Model of Nonalcoholic Steatohepatitis Induced by High-Fat Diet +CCl₄

### 1. Materials and Methods

Mold feed: high fat feed (88% basic feed +10% lard +2% cholesterol), purchased from Beijing Keao Xieli Feed Co., Ltd., with license number SCXK (Beijing) 2019-0003 and certificate number 1112622000028380; Carbon tetrachloride (CCl₄), purchased from Shanghai Macklin Biochemical Co., Ltd., with license number C10921057; Olive Oil, purchased from Sinopharm Chemical Reagent Co., Ltd., with license number 0191008.

Animal: SPF-grade male Wistar rats weighting 160±10 g were purchased from SPF (Beijing) Biotechnology Co., Ltd., with license number SCX (Beijing) 2019-0010.

Drug: NR218 composition; Bicyclol tablets were obtained from Beijing Union Pharmaceutical Factory, with a specification of 50 mg per tablet and lot number 191116; Lovastatin capsules were obtained from Yangtze River Pharmaceutical Group, with a specification of 20 mg per capsule and lot number 19030461; Ocaliva was obtained from Hubei Jiuzhou Kangda Biotechnology Co., Ltd., with a specification of 100 g per bag and lot number 20200210.

Instruments: Full-function microplate detector, Model Synergy H1, was provided by BioTek Instruments Inc.; Multi-tissue homogenizer, Model Tissuelyser-24, was provided by Shanghai Jingxin Industrial Development Co., Ltd.; Centrifuge, Model PICO 17, was provided by Thermo Fisher Scientific; Electronic balance, Model YP10001, was provided by Shanghai Yueping Electronic Balance; Analytical Balance, Model PL602-L, was provided by Mettler Toledo; Vertical Pressure Steam Sterilizer, Model SN510C, was provided by Chongqing Amato Technology Co., Ltd.; Electrophoresis instrument, Model DYY-6C, was provided by Beijing Liuyi Instrument Factory; Low-temperature centrifuge, Model FRESCO21, was provided by Thermo Fisher Scientific.

Materials: High-density lipoprotein cholesterol (HDL-C) test kit, Item No. A112-1-1 and Lot No. 20201010, was provided by Nanjing Jiancheng Bioengineering Institute; Low-density lipoprotein cholesterol (LDL-C) test kit, Item No. A113-1-1 and Lot No. 20201010, was provided by Nanjing Jiancheng Bioengineering Institute; Glutamic-pyruvic Transaminase (ALT/GPT) test kit, Item No. C009-2-1 and Lot No. 20200912, was provided by Nanjing Jiancheng Bioengineering Institute; Glutamic-oxalic Aminotransferase (AST/GOT) test kit, Item No. C010-2-1 and Lot No. 20200929, was provided by Nanjing Jiancheng Bioengineering Institute; Uric acid (UA) test kit, Item No. C012-2, Lot No. 20200907, was provided by Nanjing Jiancheng Bioengineering Institute; Free fatty acid (NEFA) test kit, Item No. A042-2-1 and Lot No. 20201010, was provided by Nanjing Jiancheng Bioengineering Institute; Blood Glucose (FPG) test kit, Item No. SH152W and Lot No. 20201016, was provided by G-clone Biotechnology Co., Ltd.; Total cholesterol (TC) test kit, Item No. A111-1-1 and Lot No. 20200825, was provided by Nanjing Jiancheng Bioengineering Institute; Triglyceride (TG) test Kit, Item No. A110-1-1 and Lot No. 20200825, Nanjing Jiancheng Bioengineering Institute; Rat Fasting Insulin (FINS) ELISA kit, Item No. ml302840, was provided by Good elisakit producers Co., Ltd.; Rat Type III Procollagen (PC-III) test kit, Item No. m1038007, was provided by Good elisakit producers Co., Ltd.; Rat laminin (LN) test kit, Item No. ml823654, was provided by Good elisakit producers Co., Ltd.; Rat Type IV collagen (IVC) test kit, Item No. m1038234, was provided by Good elisakit producers Co., Ltd.; Rat hyaluronic acid (HA) test kit, Item No. m1852321, was provided by Good elisakit producers Co., Ltd.; SIRT1 Antibody, Item No. 9475S, was provided by Cell Signaling technology Co., Ltd.; AMPKα1 Antibody, Item No. 10929-2-AP, was provided by Proteinteck; NF-κB p65 Antibody, Item No. 10745-1-AP, was provided by Proteinteck Inc.; Nrf2 Antibody, Item No. 16396-1-AP, was provided by Proteinteck Inc.; GAPDH Antibody, Item No. 10494-1-AP, was provided by Proteinteck Inc.

### 2. Methods

### (1) Reproduction and Administration of a Rat Model of Nonalcoholic Steatohepatitis Induced by a High-Fat Diet and CCl₄

A total of one hundred and twenty male Wistar rats (SPF grade, weighing 160±10 g) were randomly assigned to eight groups, including the normal control group, model group, Bicyclol group, Lovastatin group, Ocaliva group, and NR218 high (90 mg/kg), medium (30 mg/kg), and low (10 mg/kg) dose groups with fifteen rats in each. The normal control group were fed a standard diet while the other groups were exclusively fed a high-fat diet consisting of 88% basal diet +10% lard +2% cholesterol for the initial six weeks. Starting from the seventh week, they received subcutaneous injections of a 30% CCl4 olive oil solution at a dosage of 0.2 ml/100 g behind their back twice per week for a total of four times [24]. Once the model was successfully replicated, the corresponding drugs were administered orally (10 ml/kg) in each treatment group, and both the normal control group and the model group received equal volumes of a 0.5% CMC-Na solution once daily for ten consecutive weeks. Throughout the experimental period, the rats were provided with ad libitum access to food and water, while the animal chamber was maintained under controlled conditions of quietness, natural lighting, a temperature of 25±0.5° C., and a humidity level of 55±5%. The rats' body weights were measured once per week and recorded.

### (2) Collection and Analysis of Samples

### \textcircled{1} Survey on the General Condition

The weight changes of rats in each group were compared by conducting weekly weigh-ins after the experiment.

### \textcircled{2} Evaluation of Liver Indices

After blood sampling from the abdominal aorta of rats, the liver tissue was isolated, rinsed with normal saline solution, dried using filter paper, weighed accurately, and subsequently calculated the Liver indices: Liver indices=wet liver weight/body weight×100%.

\textcircled{3} Revealing Serum Biochemical Markers Associated with Nonalcoholic Steatohepatitis

At the end of the tenth week of administration, all rats underwent a 16-hour fasting period following their final administration and were intraperitoneally injected with a 20% ulinastatin solution (10 ml/kg) for anesthesia. Subsequently, blood samples were collected from the abdominal aorta and allowed to stand for one hour, and then centrifuged at 4° C. for fifteen minutes at a speed of 3000 r/min. Finally, the upper serum was stored at −80° C. Following the instructions provided with the biochemical index detection kit, levels of HDL-C, LDL-C, NEFA, AST, ALT, UA, TC, TG, PCIII, IV-C LN and HA in rat serum were determined.

### \textcircled{4} Computation of the HOMA-IR Index

According to the kit instructions, the concentration of fasting plasma glucose (FPG) in serum was determined using colorimetry, while the concentration of fasting insulin (FINS) in serum was measured through radioimmunoassay. The homeostatic model assessment for insulin resistance index (HOMA-IR) was calculated using the formula: HOMA-IR=FPG (mmol/L)×FINS (mIU/L)/22.5.

### \textcircled{5} Histopathological Examination of Hepatic Tissue in Rats

After collecting blood samples from the abdominal aorta of rats, the liver tissue was isolated and fixed in a 10% neutral formalin solution. Following standard procedures for embedding, sectioning, dewaxing, staining, clearing, and sealing, the liver tissues were subjected to HE (hematoxylin-eosin), Masson's trichrome, and oil red staining techniques respectively. Subsequently, a microscopic examination was performed to observe the pathological alterations in the liver tissues across different groups. The NASH-CRN scoring system was utilized for the assessment of NAS and liver fibrosis grade, while HALO analysis software was employed to analyze the proportion of liver fibrosis area.

\textcircled{6} Detection of Proteins Associated with Non-Alcoholic Steatohepatitis

Immunohistochemical staining: The liver tissue sections, embedded in paraffin with a thickness of 5 μm, were incubated with the target antibody for 90 minutes. Subsequently, they were incubated with a peroxidase-conjugated secondary antibody for 30 minutes. Following this, the sections were incubated with streptavidin-peroxidase-biotin complex at room temperature for 20 minutes. After chromogenic staining, the liver tissue sections were observed and analyzed under a microscope.

Western blotting experiment: Under aseptic conditions, approximately 80 mg of liver tissue was collected from each rat and liver tissue protein was extracted using a high-efficiency RIPA tissue rapid cleavage solution and PMSF. Then quantitative detection of the extracted liver tissue protein was performed using the BCA Protein Assay Kit, following the provided instructions. The expression changes of AMPK, SIRT1, NF-κB, Nrf2, and GAPDH proteins were detected according to standard procedures for Western blotting experiments. ImageJ 1.48 software was utilized for quantitative analysis.

### (3) Statistical Analysis and Processing

The experimental results were expressed as mean±standard deviation (X̅±SD). One-way analysis of variance was performed using SPSS 17.0 statistical software. Post-experiment, the LSD test was conducted to compare the two groups, with P<0.05 indicating a significant difference and P<0.01 indicating an extremely significant difference. GraphPad Prism software version 8.0 was utilized for data visualization.

### 3. Results

(1) Alterations in the Body Weight of Rat Cohorts within Each Experimental Group

As shown in Table 7, after conducting statistical analysis on the weekly weight of rats, it was observed that the weight of rats in each group exhibited a consistent increase during the pre-administration feeding period. However, following administration, there was a significant deceleration in the rate of weight gain among rats. Furthermore, when compared to the model group, all NR218 groups (low-dose, medium-dose, and high-dose) displayed a downward trend in rat weight. However, these differences were not statistically significant.

TABLE 7
Changes in body weight of rats in each group (X̅ ± SD, n = 15)

| | | | | | | NR218 | NR218 | NR218 |
|---|---|---|---|---|---|---|---|---|
| | Normal | | | | | high- | medium- | low- |
| Time | control | Model | Ocaliva | Lovastatin | Bicyclol | dose | dose | dose |
| (weeks) | group | group | group | group | group | group | group | group |
| 1 | 195.63 ± 7.32 | 196.54 ± 7.28 | 192.12 ± 5.16 | 195.70 ± 6.24 | 195.81 ± 5.12 | 195.91 ± 7.34 | 196.18 ± 5.96 | 194.18 ± 5.90 |
| 2 | 268.09 ± 9.23 | 259.59 ± 10.24 | 251.85 ± 11.78 | 253.01 ± 9.37 | 252.07 ± 9.62 | 258.31 ± 12.10 | 254.89 ± 14.05 | 261.93 ± 11.18 |
| 3 | 337.63 ± 15.41 | 324.53 ± 16.79 | 316.12 ± 16.31 | 315.37 ± 15.71 | 293.51 ± 34.27 | 304.47 ± 17.67 | 308.03 ± 30.44 | 320.98 ± 14.84 |
| 4 | 386.65 ± 21.41 | 367.20 ± 21.24 | 359.50 ± 15.40 | 368.78 ± 19.25 | 366.45 ± 19.21 | 353.75 ± 17.87 | 362.49 ± 27.00 | 366.96 ± 18.26 |
| 5 | 424.22 ± 26.68 | 406.99 ± 29.09 | 389.95 ± 30.78 | 409.85 ± 24.64 | 404.78 ± 18.71 | 389.09 ± 19.35 | 400.85 ± 30.23 | 404.82 ± 23.42 |
| 6 | 441.47 ± 34.79 | 442.93 ± 36.82 | 432.14 ± 23.79 | 439.81 ± 28.73 | 438.13 ± 24.81 | 415.29 ± 24.01 | 434.58 ± 34.77 | 442.45 ± 26.89 |
| 7 | 478.45 ± 34.83 | 472.49 ± 36.63 | 462.23 ± 28.00 | 470.75 ± 34.51 | 471.76 ± 26.05 | 452.33 ± 21.49 | 470.25 ± 37.75 | 476.20 ± 28.56 |
| 8 | 508.87 ± 41.86 | 497.37 ± 47.55 | 487.97 ± 30.51 | 491.21 ± 35.35 | 493.73 ± 28.93 | 476.81 ± 24.29 | 497.90 ± 41.54 | 502.69 ± 33.67 |
| 9 | 531.85 ± 44.95 | 483.64 ± 72.57 | 487.63 ± 39.07 | 491.46 ± 34.37 | 500.11 ± 35.71 | 478.29 ± 37.61 | 503.77 ± 50.08 | 514.39 ± 35.30 |
| 10 | 535.59 ± 49.27 | 523.34 ± 48.88 | 505.41 ± 41.23 | 503.05 ± 37.51 | 516.75 ± 35.02 | 488.73 ± 25.13 | 519.57 ± 51.10 | 528.86 ± 35.85 |
| 11 | 550.83 ± 54.81 | 515.07 ± 58.74 | 493.66 ± 55.42 | 486.62 ± 44.00 | 483.11 ± 32.67 | 479.25 ± 25.61 | 502.03 ± 44.13 | 511.16 ± 37.30 |
| 12 | 571.75 ± 47.75 | 530.49 ± 59.46 | 509.99 ± 49.38 | 506.22 ± 46.50 | 486.89 ± 36.07 | 493.19 ± 21.60 | 516.63 ± 51.24 | 529.59 ± 39.95 |
| 13 | 579.57 ± 50.19 | 555.75 ± 50.95 | 532.39 ± 48.08 | 530.00 ± 54.43 | 508.69 ± 60.33 | 496.22 ± 35.89 | 538.63 ± 47.53 | 541.35 ± 33.05 |
| 14 | 569.49 ± 59.64 | 560.46 ± 53.89 | 532.75 ± 52.06 | 531.16 ± 48.26 | 526.54 ± 51.97 | 493.27 ± 44.54 | 552.03 ± 56.18 | 533.67 ± 33.12 |
| 15 | 550.12 ± 63.80 | 544.61 ± 56.01 | 498.86 ± 46.37 | 525.60 ± 53.49 | 536.32 ± 44.50 | 494.10 ± 34.33 | 551.10 ± 58.02 | 526.87 ± 42.13 |
| 16 | 558.30 ± 61.03 | 542.47 ± 63.33 | 493.95 ± 43.25 | 539.63 ± 46.78 | 559.17 ± 38.21 | 509.77 ± 28.66 | 560.04 ± 68.91 | 547.52 ± 44.24 |
| 17 | 570.53 ± 61.76 | 560.94 ± 67.11 | 515.43 ± 41.21 | 555.78 ± 49.24 | 573.81 ± 44.40 | 528.36 ± 29.08 | 569.98 ± 73.10 | 569.73 ± 38.59 |
| 18 | 579.63 ± 61.52 | 565.87 ± 62.67 | 525.15 ± 42.35 | 561.12 ± 51.49 | 581.09 ± 46.08 | 534.35 ± 27.63 | 559.42 ± 70.02 | 574.85 ± 36.54 |

### (2) Effects of NR218 on a Hepatic Index in a Rat Model of Nonalcoholic Steatohepatitis Induced by a High-Fat Diet and CCl₄

As shown in Table 8, through the investigation of the liver indices in each group, it was observed that the model group exhibited a significant increase compared to the normal control group (P<0.01). Furthermore, administration of low, medium and high doses of NR218 resulted in a significant reduction in liver indices when compared to the model group (P<0.01). These findings strongly indicate that NR218 effectively inhibits liver hypertrophy in rats with nonalcoholic steatohepatitis induced by a high-fat diet and CCl₄, surpassing the efficacy of positive drugs.

TABLE 8
Liver indices of rats in each group (X̅ ± SD)

| Groups | Sample Size | Liver Indices (%) |
|---|---|---|
| Normal control group | 15 | 2.36 ± 0.20 |
| Model group | 15 | 3.78 ± 0.73^{##} |
| Bicyclic alcohol positive control group | 15 | 3.48 ± 0.41* |
| Lovastatin positive control group | 15 | 3.56 ± 0.49 |
| Obercholic acid positive control group | 15 | 3.57 ± 0.31 |
| NR218 low-dose group | 15 | 2.52 ± 0.28** |
| NR218 medium-dose group | 15 | 2.44 ± 0.27** |
| NR218 high-dose group | 15 | 2.39 ± 0.20** |

Note:
^{##}P < 0.01 compared to the normal control group; **P < 0.01 compared to the model group.

(3) Effects of NR218 on Serum Parameters in Rats with Non-Alcoholic Steatohepatitis Induced by a High-Fat Diet and CCl₄

According to the instructions of the biochemical index detection kit, serum levels of AST, ALT, HDL-C, LDL-C, UA, NEFA, TC, TG, FPG, FINS, PCIII, IV-C, LN and HA were measured. As shown in Table 9 and 10, the experimental results demonstrated that rats in the model group compared to the normal control group, exhibited significantly elevated levels of serum ALT, AST, TC, TG, LDL-C, UA, PCIII, IV-C, LN and HA (P<0.01), along with a significant decrease in HDL-C level (P<0.01), indicating successful replication of the model. In comparison to the model group, NR218 at high, medium and low doses significantly reduced serum levels of AST, ALT, LDL-C, UA, NEFA, TC, TG, PCIII, IV-C, LN and HA in rats (P<0.01 or P<0.05), increased HDL-C content significantly (P<0.01 or P<0.05), and effectively improved hepatic function as well as blood lipid levels and liver fibrosis in rats with nonalcoholic steatohepatitis.

TABLE 9
Effects of NR218 on serum biochemical indices of rats with nonalcoholic steatohepatitis (X̅ ± SD, n = 15)

| | ALT | AST | TC | TG | HDL-C | LDL-C | UA | NEFA | HOMA- |
|---|---|---|---|---|---|---|---|---|---|
| Groups | (U/L) | (U/L) | (mmol/L) | (mmol/L) | (mmol/L) | (mmol/L) | (mol/L) | (mmol/L) | IR |
| Normal | 16.26 ± | 44.54 ± | 1.36 ± | 0.45 ± | 2.33 ± | 0.31 ± | 84.74 ± | 0.32 ± | 3.39 ± |
| control | 5.81 | 10.85 | 0.67 | 0.13 | 0.97 | 0.20 | 16.80 | 0.10 | 0.60 |
| group | | | | | | | | | |
| Model | 91.25 ± | 98.39 ± | 3.34 ± | 1.30 ± | 1.05 ± | 1.79 ± | 147.91 ± | 1.07 ± | 6.81 ± |
| group | 26.62^{##} | 17.17^{##} | 1.45^{##} | 0.46^{##} | 0.51^{##} | 0.33^{##} | 40.70^{##} | 0.21^{##} | 1.55^{##} |
| Ocaliva | 54.91 ± | 62.32 ± | 1.76 ± | 0.50 ± | 1.30 ± | 1.29 ± | 106.41 ± | 0.69 ± | 4.90 ± |
| group | 15.32** | 13.11** | 1.76** | 0.12** | 0.57 | 0.31** | 26.89** | 0.17** | 1.50** |
| Lovastatin | 43.66 ± | 69.18 ± | 1.76 ± | 0.61 ± | 1.31 ± | 1.20 ± | 118.58 ± | 0.76 ± | 5.50 ± |
| group | 24.63** | 19.20** | 1.76** | 0.26** | 0.80 | 0.25** | 42.61* | 0.18** | 1.23** |
| Bicyclol | 44.39 ± | 66.96 ± | 1.85 ± | 0.63 ± | 1.29 ± | 0.90 ± | 125.89 ± | 0.77 ± | 5.48 ± |
| group | 19.83** | 6.13** | 0.82** | 0.12** | 0.68 | 0.39** | 39.39 | 0.30** | 1.00** |
| NR218 | 30.55 ± | 57.59 ± | 2.18 ± | 0.62 ± | 2.18 ± | 0.79 ± | 94.09 ± | 0.43 ± | 4.23 ± |
| high- | 12.37** | 12.60** | 0.88** | 0.16** | 1.10** | 0.25** | 28.22** | 0.12** | 0.72** |
| dose | | | | | | | | | |
| group | | | | | | | | | |
| NR218 | 37.01 ± | 62.00 ± | 2.44 ± | 0.73 ± | 1.90 ± | 0.91 ± | 98.94 ± | 0.52 ± | 4.79 ± |
| medium- | 17.00** | 14.49** | 0.99* | 0.24** | 0.90** | 0.34** | 13.64** | 0.16** | 0.80** |
| dose | | | | | | | | | |
| group | | | | | | | | | |
| NR218 | 42.67 ± | 66.80 ± | 2.63 ± | 0.81 ± | 1.76 ± | 1.16 ± | 109.37 ± | 0.64 ± | 5.13 ± |
| low- | 13.26** | 11.83** | 0.91* | 0.18** | 0.96* | 0.49** | 42.05** | 0.15** | 1.11** |
| dose | | | | | | | | | |
| group | | | | | | | | | |

Note:
Compared to the normal control group, denoted by ^{#}P < 0.05 and ^{##}P < 0.01;
Compared to the model group, denoted by *P < 0.05 and **P < 0.01.

TABLE 10
Effects of NR218 on four indices of serum hepatic fibrosis in rats (X̅ ± SD, n = 15)

| Groups | HA (μg/L) | LN (μg/L) | PC-III (μg/L) | IV-C (μg/L) |
|---|---|---|---|---|
| Normal control group | 16.95 ± 2.85 | 55.30 ± 12.58 | 4.59 ± 0.90 | 4.73 ± 1.33 |
| Model group | 62.13 ± 21.36^{##} | 135.59 ± 51.60^{##} | 11.84 ± 2.20^{##} | 18.06 ± 5.43^{##} |
| Bicyclol positive control group | 45.78 ± 22.99 | 110.64 ± 44.60 | 8.78 ± 1.87 | 13.22 ± 4.50 |
| Lovastatin positive control group | 47.85 ± 19.06* | 117.50 ± 43.83 | 10.52 ± 2.34* | 15.59 ± 7.47 |
| Ocaliva positive control group | 50.19 ± 17.94* | 118.43 ± 39.88* | 10.82 ± 1.64** | 15.40 ± 7.08* |
| Z018 high-dose group | 26.47 ± 14.29** | 67.68 ± 14.72** | 5.47 ± 1.08** | 7.94 ± 2.07** |
| Z018 medium-dose group | 32.36 ± 10.42** | 73.80 ± 13.13** | 6.45 ± 1.40** | 8.78 ± 2.76** |
| Z018 low-dose group | 42.88 ± 26.82** | 85.22 ± 18.78** | 7.07 ± 1.48** | 11.07 ± 3.54** |

Note:
Compared to the normal control group, denoted by *P < 0.05 and ^{##}P < 0.01; Compared to the model group, denoted by *P < 0.05 and **P < 0.01.

(4) Effects of NR218 on the Hepatic Histopathological Changes in Rats with Nonalcoholic Steatohepatitis Induced by a High-Fat Diet and CCl₄

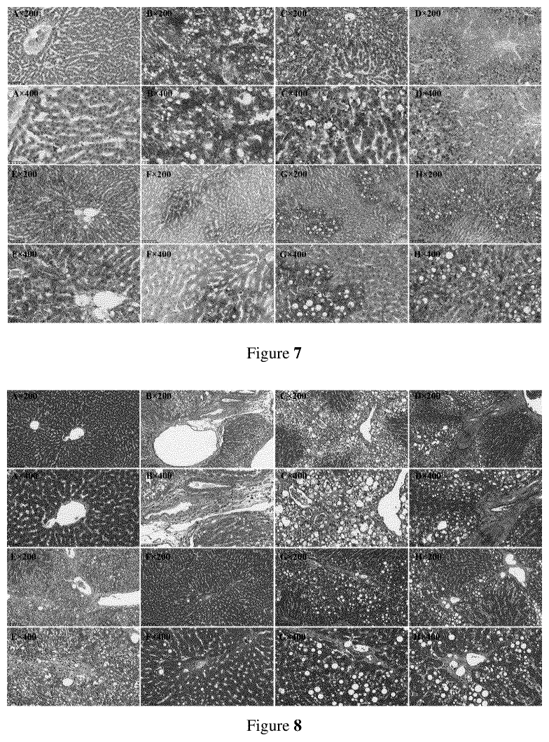

\textcircled{1} As shown in FIG. 6, the HE staining results revealed that the liver cells of rats in the normal control group exhibited a well-organized arrangement, clear hepatic lobular structure, and absence of steatosis or inflammatory cell infiltration. Conversely, rats in the model group displayed swollen liver cells with prominent lipid droplet voids, balloon-like degeneration, significant enlargement of hepatic sinuses, conspicuous inflammatory cell infiltration, and notable increase in liver collagen content, indicating successful establishment of the model. Compared to the normal control group, administration of low, medium and high doses of NR218 exhibited varying degrees of reduction in hepatocytic fat granules and liver tissue collagen content. Moreover they significantly alleviated inflammatory cell infiltration and hepatocyte necrosis, surpassing the effects observed with positive drugs Ocaliva, Lovastatin and Dicyclol.

\textcircled{2} As shown in FIG. 7, the Oil red O staining results revealed that the liver cell nuclei of rats in the normal control group exhibited light staining, devoid of any red-stained lipid droplets. In the model group, hepatocytes displayed swelling and a substantial number of large blister-like red lipid droplets were observed within the cytoplasm, some of which coalesced into larger droplets while most were distributed in a flaky pattern. Hepatocytes exhibited irregular arrangement and loss of cytoskeletal integrity. Comparatively, scattered small lipid droplets with the occasional presence of large ones could be seen in the liver cells of rats treated with Dicyclol, Lovastatin and Ocaliva positive control group as well as NR218 high-dose, medium-dose, and low-dose groups. These treatments significantly ameliorated steatosis severity and reduced lipid droplet content. Notably, NR218 demonstrated superior efficacy at each dosage compared to Dicyclol, Lovastatin and Ocaliva.

\textcircled{3} As shown in FIG. 8, the results of Masson staining demonstrated that rats exhibited a significant increase in collagen content within the liver tissues of rats in the model group, with predominant deposition presented a mesh-like pattern in the perisinusoidal and portal areas. Furthermore, rats treated with NR218 at high, medium, and low doses resulted in a remarkable reduction in liver tissue fibrosis compared to the model group, while the mesh-like collagen deposition disappeared following NR218 administration. Notably, all doses of NR218 demonstrated superior efficacy compared to Dicyclol, Lovastatin and Ocaliva.

(5) Effects of NR218 on the Expression of Relevant Proteins in Hepatic Tissue of Rats with Non-Alcoholic Steatohepatitis

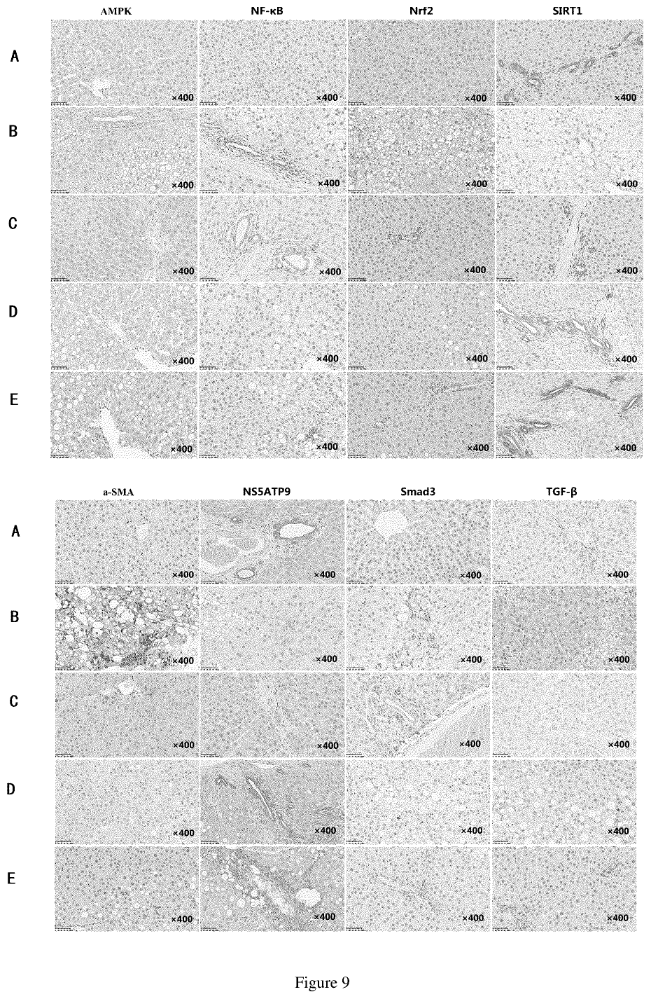

In order to further investigate the potential biological mechanism of NR218 in treating nonalcoholic steatohepatitis induced by high-fat diet and CCl4, the immunohistochemical methods to detect the expressions of Smad, TGFβ, α-SMA and NS5ATP9 proteins in rat liver tissues were utilized. As shown in FIG. 9, the results demonstrated that treatment with high, medium, and low doses of NR218 could effectively reduce the protein expression levels of Smad, TGF-β, and a-SMA in the liver tissue of rats with non-alcoholic steatohepatitis to varying degrees while significantly increasing the protein expression level of NS5ATP9. Additionally, western blotting and immunohistochemistry were employed to investigate changes in protein expression levels of AMPK, SIRT1, Nrf2, and NF-κB in rat liver tissue. As shown in Table 11, the results demonstrated that the hepatic tissue of rats exhibited a significant decrease in protein expression levels of AMPK and SIRT1 (P<0.01) in the model group, which compared to the normal control group, while there was a substantial increase in protein expression levels of NF-κB and Nrf2 (P<0.01). In contrast to the model group, treatment with NR218 at high, medium, and low doses resulted in a significant increase in AMPK and SIRT1 protein expression levels (P<0.01 or P<0.05), accompanied by a significant decrease in NF-κB and Nrf2 protein expression levels (P<0.01). Notably, the high-dose group exhibited superior efficacy. The above findings suggest that NR218 may potentially mitigate nonalcoholic steatohepatitis by modulating inflammation, oxidative stress, and liver fibrosis through the AMPK/SIRT1/NF-κB/Smad signaling pathways.

TABLE 11
Effects of NR218 on the expression levels of AMPK, SIRT1, Nrf2 and NF-
κB in hepatic tissues of rats (X̅ ± SD)

| Groups | Sample size | AMPK | SIRT1 | Nrf2 | NF-κB |
|---|---|---|---|---|---|
| Normal control group | 3 | 1.23 ± 0.09 | 1.27 ± 0.26 | 0.88 ± 0.09 | 1.01 ± 0.04 |
| Model group | 3 | 0.44 ± 0.07^{##} | 0.40 ± 0.08^{##} | 1.66 ± 0.01^{##} | 1.74 ± 0.23^{##} |
| NR218 high-dose group | 3 | 1.30 ± 0.12** | 1.27 ± 0.18** | 0.76 ± 0.08** | 0.50 ± 0.17** |
| NR218 medium-dose group | 3 | 1.18 ± 0.10** | 1.13 ± 0.15** | 0.79 ± 0.14** | 0.75 ± 0.04** |
| NR218 low-dose group | 3 | 0.83 ± 0.22* | 0.91 ± 0.28* | 0.93 ± 0.14** | 1.02 ± 0.03** |

Note:
Compared to the normal control group, denoted by ^{##}P < 0.01; Compared to the model group, denoted by *P < 0.05 and **P < 0.01.

### 4. Conclusion

The present study utilized a high-fat diet combined with CCl4 to establish a rat model of nonalcoholic steatohepatitis (NASH). Through comprehensive evaluation of the rats' general condition (body weight and liver weight), serum biochemical markers, and histopathology, it was observed that NR218 significantly ameliorated hepatic steatosis and fibrosis in rats with nonalcoholic steatohepatitis. The liver fibrosis grade score, and liver fibrosis area ratio in NASH rats, while preventing disease progression. Additionally, NR218 decreased the Liver indices as well as blood lipid levels and improved insulin resistance in NASH rats. In terms of the protein expression levels, NR218 markedly upregulated the levels of AMPK, NS5ATP9, and SIRT1 proteins while downregulating NF-κB, Nrf2, Smad, TGF-β, and a-SMA proteins in rat liver tissues. These above findings collectively suggest that NR218 exerts significant therapeutic effects on high-fat diet and CCl4 induced nonalcoholic steatohepatitis may be associated with the modulation of the inflammatory and antioxidant responses via the AMPK/SIRT1 pathway as well as attenuation of liver fibrosis through Smad signaling.

1. A composition of small molecular compounds, namely licochalcone A, licochalcone C, licochalcone D, licochalcone E, formononetin, glabrone and licoflavine C, wherein the composition functions as a negative regulator in the Nrf2 signaling pathway.

2. The composition according to claim 1, wherein the weight ratio of licochalcone A, licochalcone C, licochalcone D, licochalcone E, formononetin, glabrone and licoflavine C is 60˜75:2˜6:1˜4:5˜10:1˜5:1˜5:1˜5.

3. The composition according to claim 1, wherein the weight ratio of licochalcone A, licochalcone C, licochalcone D, licochalcone E, formononetin, glabrone and licoflavine C is 65-75:3˜5:1˜3:6˜8:2˜3:2˜3:1˜2.

4. The composition according to claim 1, wherein the composition exhibits a negative regulatory effect on the expression of the Nrf2 gene during the occurrence and development of nonalcoholic fatty liver disease.

5. The composition according to claim 1, wherein the licochalcone A, licochalcone C, licochalcone D, licochalcone E, formononetin, glabrone and licoflavine C derived from botanical sources or synthesized.

6. A preparation, comprising the composition of claim 1, and pharmaceutically or food-acceptable excipients.

7. A method for negatively regulating the Nrf2 signaling pathway, comprising applying the composition of claim 1.

8. A method for treating non-alcoholic fatty liver disease (NAFLD), comprising administering a pharmaceutical composition comprising the composition of claim 1.

9. The method of claim 8, wherein the non-alcoholic fatty liver disease is a condition characterized by non-alcoholic steatohepatitis (NASH).

10. The method of claim 8, wherein the non-alcoholic fatty liver disease is characterized by dysregulation in glycolipid metabolism and can lead to liver fibrosis, cirrhosis, or hepatocellular carcinoma.
